(19) United States
(12) Patent Application Publication
(10) Pub. No.: US 2013/0303560 A1
(43) Pub. Date: Nov. 14, 2013

(54) Combination

(71) Applicants: Rakesh Kumar, Collegeville, PA (US); Robert S. Kerbel, Toronto (CA)

(72) Inventors: Rakesh Kumar, Collegeville, PA (US); Robert S. Kerbel, Toronto (CA)

(73) Assignees: SUNNYBROOK RESEARCH INSTITUTE, Toronto (CA); GLAXOSMITHKLINE INTELLECTUAL PROPERTY LIMITED, Brentford, Middlesex (GB)

(21) Appl. No.: 13/980,904

(22) PCT Filed: Jan. 31, 2012
(86) PCT No.: PCT/US12/23261

§ 371 Date: Jul. 22, 2013

Related U.S. Application Data
(60) Provisional application No. 61/438,426, filed on Feb. 1, 2011.

(51) Int. Cl. A61K 31/506; A61K 31/4745

(52) U.S. Cl. CPC A61K 31/506; A61K 31/4745
USPC 514/275

(57) ABSTRACT

The present invention relates to a method of treating breast cancer in a human and to pharmaceutical combinations useful in such treatment. In particular, the method relates to a breast cancer treatment method that includes administering 5-[[4-[(2,3-dimethyl-2H-indazol-6-yl)methylamino]-2-pyrimidinyl]amino]-2-methylbenzenesulfonamide, or a pharmaceutically acceptable salt thereof, and (S)-10-[(dimethylamino)methyl]-4-ethyl-4,9-dihydroxy-1H-pyrano[3′,4′:6,7]indolizino[1,2-b]quinoline-3,14-(4H,12H)-dione, or a pharmaceutically acceptable salt thereof, to a human in need thereof.

### FIELD OF THE INVENTION

The present invention relates to a method of treating cancer in a mammal and to combinations useful in such treatment. In particular, the method relates to a novel combination comprising a VEGFR inhibitor and a topoisomerase inhibitor, pharmaceutical compositions comprising the same, and methods of using such combinations in the treatment of cancer.

### BACKGROUND OF THE INVENTION

Generally, cancer results from the deregulation of the normal processes that control cell division, differentiation and apoptotic cell death. Apoptosis (programmed cell death) plays essential roles in embryonic development and pathogenesis of various diseases, such as degenerative neuronal diseases, cardiovascular diseases and cancer. One of the most commonly studied pathways, which involves kinase regulation of apoptosis, is cellular signaling from growth factor receptors at the cell surface to the nucleus (Crews and Erikson, Cell, 74:215-17, 1993).

The process of angiogenesis is the development of new blood vessels from the pre-existing vasculature. Angiogenesis is defined herein as involving: (i) activation of endothelial cells; (ii) increased vascular permeability; (iii) subsequent dissolution of the basement membrane and extravasation of plasma components leading to formation of a provisional fibrin gel extracellular matrix; (iv) proliferation and mobilization of endothelial cells; (v) reorganization of mobilized endothelial cells to form functional capillaries; (vi) capillary loop formation; and (vi) deposition of basement membrane and recruitment of perivascular cells to newly formed vessels. Normal angiogenesis is active during tissue growth from embryonic development through maturity and then enters a period of relative quiescence during adulthood. Normal angiogenesis is also activated during wound healing, and at certain stages of the female reproductive cycle. Inappropriate or pathological angiogenesis has been associated with several disease states including various retinopathies, ischemic disease, atherosclerosis, chronic inflammatory disorders, and cancer. The role of angiogenesis in disease states is discussed, for instance, in Fan et al., Trends in Pharmacol Sci. 16:54-66; Shawver et al., DDT Vol. 2, No. 2 February 1997; Folkmann, 1995, Nature Medicine 1:27-31.

In cancer the growth of solid tumors has been shown to be dependent on angiogenesis. The progression of leukemias as well as the accumulation of fluid associated with malignant ascites and pleural effusions also involve pro-angiogenic factors. (See Folkmann, J., J. Nat'l. Cancer Inst, 1990, 82, 4-6).

Central to the process of angiogenesis are vascular endothelial growth factor (VEGF) and its receptors, termed vascular endothelial growth factor receptor(s) (VEGFRs). The roles VEGF and VEGFRs play in the vascularization of solid tumors, progression of hematopoietic cancers and modulation of vascular permeability have drawn great interest in the scientific community. VEGF is a polypeptide, which has been linked to inappropriate or pathological angiogenesis (Pinedo, H. M. et al. The Oncologist, Vol.5, No. 90001, 1-2, Apr. 2000). VEGFR(s) are protein tyrosine kinases (PTKs) that catalyze the phosphorylation of specific tyrosine residues in proteins that are involved in the regulation of cell growth, differentiation, and survival. (A. F. Wilks, Progress in Growth Factor Research, 1990, 2, 97-111; S. A. Courtneidge, Dev. Supp.1, 1993, 57-64; J. A. Cooper, Semin. Cell Biol., 1994,5(6),377-387; R. F. Paulson, Semin. Immunol. 1995, 7(4), 267-277; A. C. Chan, Curr. Opin. Immunol. 1996, 8(3), 394-401).

Three PTK receptors for VEGF have been identified: VEGFRI (Flt-l); VEGFR2 (Flk-I and KDR) and VEGFR3 (Flt-4). These receptors are involved in angiogenesis and participate in signal transduction. (Mustonen, T. et al. J. Cell. Biol. 1995: 129:895-898; Ferrara and Davis-Smyth, Endocrine Reviews, 18(1):4-25, 1997; McMahon, G., The Oncologist, Vol. 5, No 90001, 3-10, Apr. 2000).

Of particular interest is VEGFR2, which is a transmembrane receptor PTK expressed primarily in endothelial cells. Activation of VEGFR-2 by VEGF is a critical step in the signal transduction pathway that initiates tumor angiogenesis. VEGF expression may be constitutive to tumor cells and can also be upregulated in response to certain stimuli. One such stimulus is hypoxia, where VEGF expression is upregulated in both tumor and associated host tissues. The VEGF ligand activates VEGFR2 by binding to its extracellular VEGF binding site. This leads to receptor dimerization of VEGFRs and autophosphorylation of tyrosine residues at the intracellular kinase domain of VEGFR2. The kinase domain operates to transfer a phosphate from ATP to the tyrosine residues, thus providing binding sites for signaling proteins downstream of VEGFR-2 leading ultimately to angiogenesis. (Ferrara and Davis-Smyth, Endocrine Reviews, 18(1):4-25, 1997; McMahon, G. The Oncologist, Vol. 5, No. 90001, 3-10, Apr. 2000.)

Consequently, antagonism of the VEGFR2 kinase domain would block phosphorylation of tyrosine residues and serve to disrupt initiation of angiogenesis. Specifically, inhibition at the ATP binding site of the VEGFR2 kinase domain would prevent binding of ATP and prevent phosphorylation of tyrosine residues. Such disruption of the proangiogenesis signal transduction pathway associated with VEGFR2 should therefore inhibit tumor angiogenesis and thereby provide a potent treatment for cancer or other disorders associated with inappropriate angiogenesis. Votrient (pazopanib hydrochloride) is a multi-tyrosine kinase inhibitor of vascular endothelial growth factor receptor (VEGFR)-1, VEGFR-2, VEGFR-3, platelet-derived growth factor receptor (PDGFR)-α and -β, fibroblast growth factor receptor (FGFR)-1 and -3, cytokine receptor (Kit), interleukin-2 receptor inducible T-cell kinase (Itk), leukocyte-specific protein tyrosine kinase (Lck), and transmembrane glycoprotein receptor tyrosine kinase (c-Fms) and is approved in the US for the treatment of patients with advanced renal cell carcinoma. The chemical name of pazopanib hydrochloride is 5-[[4-[(2,3-dimethyl-2H-indazol-6-yl)methylamino]-2-pyrimidinyl]amino]-2-methylbenzenesulfonamide monohydrochloride.

The structure of the DNA helix within eukaryotic cells imposes certain topological problems that the cellular apparatus must solve in order to use its genetic material as a template. The separation of the DNA strands is fundamental to cellular processes such as DNA replication and transcription. Since eukaryotic DNA is organized into chromatin by chromosomal proteins, the ends are constrained and the strands cannot unwind without the aid of enzymes that alter topology. It has long been recognized that the advancement of the transcription or replication complex along the DNA helix would be facilitated by a swivel point which would relieve the torsional strain generated during these processes. Topoisomerases are enzymes that are capable of altering DNA topology in eukaryotic cells. They are critical for important cellular functions and cell proliferation.

There are two classes of topoisomerases in eukaryotic cells, type I and type II. Topoisomerase I is a monomeric enzyme of approximately 100,000 molecular weight. The enzyme binds to DNA and introduces a transient single strand break, unwinds the double helix (or allows it to unwind), and subsequently reseals the break before dissociating from the DNA strand. Topoisomerase II consists of two identical subunits of molecular weight 170,000. Topoisomerase II transiently breaks both strands of the helix and passes another double-strand segment through the break. Camptothecin is a water-insoluble, cytotoxic alkaloid produced by Camptotheca accuminata trees indigenous to China and Nothapodytes foetida trees indigenous to India. Camptothecin and a few close congeners thereof are the only class of compounds known to inhibit topoisomerase I. Inhibition of topoisomerase II is the major target of important commercial oncolytic agents (e.g., etoposide, doxorubicin and mitoxantrone) as well as other oncolytic agents still undergoing development. Camptothecin (and its known congeners) have no effect on topoisomerase II and none of the known topoisomerase II inhibitors has any significant effect on topoisomerase I. Hycamtin® (topotecan hydrochloride) is a semi-synthetic derivative of campotothecin that exhibits topoisomerase I-inhibitory activity. Hycamtin® is approved in the US for the treatment of relapsed small cell lung cancer, ovarian cancer and cervical cancer. The chemical name for topotecan hydrochloride is (S)-10-[(dimethylamino)methyI]-4-ethyl-4,9-dihydroxy-1H-pyrano[3′,4′:6,7]indolizino[1,2-b]quinoline-3,14-(4H,12H)-dione monohydrochloride.

It would be useful to provide a novel therapy which provides more effective and/or enhanced treatment of an individual suffering the effects of cancer.

### SUMMARY OF THE INVENTION

One embodiment of this invention provides a combination that includes:
(i) a compound of Structure (I):

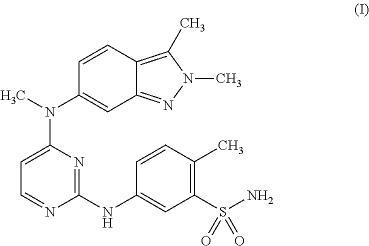 [SMILES: Cc1ccc(Nc2nccc(N(C)c3ccc4c(C)n(C)nc4c3)n2)cc1S(N)(=O)=O]

or a pharmaceutically acceptable salt thereof; and
(ii) a compound of Structure (II):

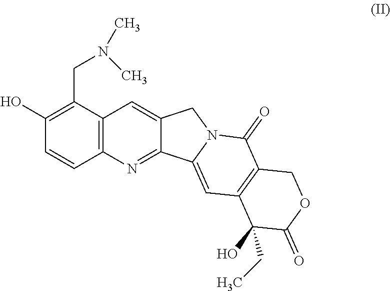 [SMILES: CC[C@@]1(O)C(=O)OCc2c1cc1n(c2=O)Cc2cc3c(CN(C)C)c(O)ccc3nc2-1]

or a pharmaceutically acceptable salt thereof.

One embodiment of this invention provides a method of treating breast cancer in a human in need thereof which comprises the in vivo administration of a therapeutically effective amount of a combination of 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, and (S)-10-[(dimethylamino)methyl]-4-ethyl-4,9-dihydroxy-1H-pyrano[3′,4′:6,7]indolizino[1,2-b]quinoline-3,14-(4H,12H)-dione, or a pharmaceutically acceptable salt, suitably the hydrochloride salt, thereof, to such human.

One embodiment of this invention provides a method of treating breast cancer in a human in need thereof which comprises the in vivo administration of a therapeutically effective amount of a combination of 54[44(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, and (S)-10-[(dimethylamino)methyl]-4-ethyl-4,9-dihydroxy-1H-pyrano[3′,4′:6,7]indolizino [1,2-b]quinoline-3,14-(4H,12H)-dione, or a pharmaceutically acceptable salt, suitably the hydrochloride salt, thereof, to such human, wherein the combination is administered within a specified period, and wherein the combination is administered for a duration of time.

One embodiment of this invention provides a method of treating breast cancer in a human in need thereof which comprises the in vivo administration of a therapeutically effective amount of a combination of 5-[[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, and (S)-10-[(dimethylamino)methyl]-4-ethyl-4,9-dihydroxy-1H-pyrano[3′,4′:6,7]indolizino [1,2-b]quinoline-3,14-(4H,12H)-dione, or a pharmaceutically acceptable salt, suitably the hydrochloride salt, thereof, to such human, wherein the compounds of the combination are administered sequentially.

### BRIEF DESCRIPTION OF THE DRAWINGS

FIG. 1 illustrates percent survival after an indicated number of days when treatment of Advanced Metastatic Breast Cancer (MDA-MB-231/LM2-4) was initiated at day 39, 19 days post primary tumor resection, for the control group, the low dose metronomic oral topotecan alone group, the maximum tolerated group, the pazopanib alone group, the low dose oral topotecan+pazopanib group, and the maximum tolerated dose topotecan+pazopanib group.

### DETAILED DESCRIPTION OF THE INVENTION

The present invention relates to combinations that exhibit antitumor activity. Suitably, the method relates to methods of treating breast cancer by the co-administration of 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof, (hereinafter Compound A, or a pharmaceutically acceptable salt, suitably the monohydrochloride salt, thereof), which compound is represented by Structure I:

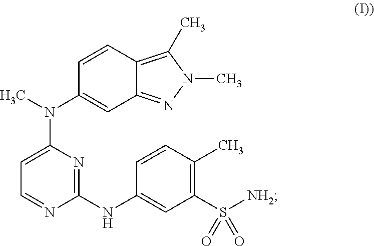 [SMILES: Cc1ccc(Nc2nccc(N(C)c3ccc4c(C)n(C)nc4c3)n2)cc1S(N)(=O)=O]

and (S)-10-[(dimethylamino)methyl]-4-ethyl-4,9-dihydroxy-1H-pyrano[3′,4′:6,7]indolizino[1,2-b]quinoline-3,14-(4H,12H)-dione, or a pharmaceutically acceptable salt, suitably the hydrochloride salt, thereof, (hereinafter Compound B or a pharmaceutically acceptable salt, suitably the hydrochloride salt, thereof), which compound is represented by Structure II:

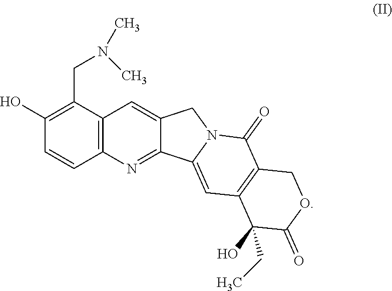 [SMILES: CC[C@@]1(O)C(=O)OCc2c1cc1n(c2=O)Cc2cc3c(CN(C)C)c(O)ccc3nc2-1]

Compound A is disclosed and claimed, along with pharmaceutically acceptable salts thereof, as being useful as an inhibitor of VEGFR activity, particularly in treatment of cancer, in International Application No. PCT/US01/49367, having an International filing date of Dec. 19, 2001, International Publication Number WO02/059110 and an International Publication date of Aug. 1, 2002, the entire disclosure of which is hereby incorporated by reference, Compound A is the compound of Example 69. Compound A can be prepared as described in International Application No. PCT/US01/49367.

Suitably, Compound A is in the form of a monohydrochloride salt. This salt form can be prepared by one of skill in the art from the description in International Application No. PCT/US01/49367, having an International filing date of Dec. 19, 2001.

Compound A is sold commercially as the monohydrochloride salt. Compound A is known by the generic name pazopanib and the trade name Votrient®.

Compound B is disclosed and claimed, along with pharmaceutically acceptable salts thereof, as being useful as an inhibitor of topoisomerase I, particularly in treatment of cancer, in U.S. Pat. No. 5,004,758, having a filing date of Nov. 2, 1988, the entire disclosure of which is hereby incorporated by reference, Compound B is compound 1S (as the acetate salt). Compound B can be prepared as described in U.S. Pat. No. 5,734,056.

Suitably, Compound B is in the form of a hydrochloride salt. The salt form can be prepared by one of skill in the art from the description in U.S. Pat. No. 5,004,758 and/or by methods that will be readily apparent to those skilled in the art.

Compound B is sold commercially as the monohydrochloride salt. Compound B is known by the generic name topotecan and the trade name Hycamtin®.

The administration of a therapeutically effective amount of the combinations of the invention are advantageous over the individual component compounds in that the combinations will provide one or more of the following improved properties when compared to the individual administration of a therapeutically effective amount of a component compound: i) a greater anticancer effect than the most active single agent, ii) synergistic or highly synergistic anticancer activity, iii) a dosing protocol that provides enhanced anticancer activity with reduced side effect profile, iv) a reduction in the toxic effect profile, v) an increase in the therapeutic window, or vi) an increase in the bioavailability of one or both of the component compounds.

The compounds of the invention may contain one or more chiral atoms, or may otherwise be capable of existing as two enantiomers. Accordingly, the compounds of this invention include mixtures of enantiomers as well as purified enantiomers or enantiomerically enriched mixtures. Also, it is understood that all tautomers and mixtures of tautomers are included within the scope of Compound A, and pharmaceutically acceptable salts thereof, and Compound B, and pharmaceutically acceptable salts thereof.

The compounds of the invention may form a solvate which is understood to be a complex of variable stoichiometry formed by a solute (in this invention, Compound A or a salt thereof and/or Compound B or a salt thereof) and a solvent. Such solvents for the purpose of the invention may not interfere with the biological activity of the solute. Examples of suitable solvents include, but are not limited to, water, methanol, ethanol and acetic acid. Suitably the solvent used is a pharmaceutically acceptable solvent. Suitably the solvent used is water.

The pharmaceutically acceptable salts of the compounds of the invention are readily prepared by those of skill in the art.

Also, contemplated herein is a method of treating breast cancer using a combination of the invention where Compound A, or a pharmaceutically acceptable salt thereof, and/or Compound B or a pharmaceutically acceptable salt thereof are administered as pro-drugs. Pharmaceutically acceptable pro-drugs of the compounds of the invention are readily prepared by those of skill in the art.

When referring to a dosing protocol, the term “day”, “per day” and the like, refer to a time within one calendar day which begins at midnight and ends at the following midnight.

By the term “treating” and derivatives thereof as used herein, is meant therapeutic therapy. In reference to a particular condition, treating means: (1) to ameliorate the condition of one or more of the biological manifestations of the condition, (2) to interfere with (a) one or more points in the biological cascade that leads to or is responsible for the condition or (b) one or more of the biological manifestations of the condition, (3) to alleviate one or more of the symptoms, effects or side effects associated with the condition or treatment thereof, or (4) to slow the progression of the condition or one or more of the biological manifestations of the condition.

Prophylactic therapy is also contemplated thereby. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing breast cancer, such as when a subject has a strong family history of breast cancer or when a subject has been exposed to a carcinogen.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

By the term “combination” and derivatives thereof, as used herein is meant either, simultaneous administration or any manner of separate sequential administration of a therapeutically effective amount of Compound A, or a pharmaceutically acceptable salt thereof, and Compound B or a pharmaceutically acceptable salt thereof. Preferably, if the administration is not simultaneous, the compounds are administered in a close time proximity to each other. Furthermore, it does not matter if the compounds are administered in the same dosage form, e.g. one compound may be administered topically and the other compound may be administered orally. Suitably, both compounds are administered orally.

As used herein the term “Compound A²” means —Compound A, or a pharmaceutically acceptable salt thereof—.

As used herein the term “Compound B²” means —Compound B, or a pharmaceutically acceptable salt thereof—.

In some embodiments according to the present invention, the combinations of this invention are administered within a “specified period”.

By the term “specified period” and derivatives thereof, as used herein is meant the interval of time between the administration of one of Compound A² and Compound B² and the other of Compound A² and Compound B². Unless otherwise defined, the specified period can include simultaneous administration. When both compounds of the invention are administered once a day the specified period refers to timing of the administration of Compound A² and Compound B² during a single day. When one or both compounds of the invention are administered more than once a day, the specified period is calculated based on the first administration of each compound on a specific day. All administrations of a compound of the invention that are subsequent to the first during a specific day are not considered when calculating the specific period.

The specified period can be various time periods. For example, Compound A² and Compound B² can be administered within about 24, 23, 22, 21, 20, 19, 18, 17, 16, 15, 14, 13, 12, 11, 10, 9, 8, 7, 6, 5, 4, 3, 2 or 1 hours of each other, in which case the specified period will be about 24, 23, 22, 21, 20, 19, 18, 17, 16, 15, 14, 13, 12, 11, 10, 9, 8, 7, 6, 5, 4, 3, 2 or 1 hours, respectively. As used herein, the administration of Compound A² and Compound B² in less than about 45 minutes apart is considered simultaneous administration.

Suitably, when the combination of the invention is administered for a “specified period,” the compounds will be co-administered for a “duration of time.”

By the term “duration of time” and derivatives thereof, as used herein is meant that both compounds of the invention are administered within a “specified period” for an indicated number of consecutive days, optionally followed by a number of consecutive days where only one of the component compounds is administered. Unless otherwise defined, the “duration of time” and in all dosing protocols described herein, do not have to commence with the start of treatment and terminate with the end of treatment, it is only required that the number of consecutive days in which both compounds are administered and the optional number of consecutive days in which only one of the component compounds is administered, or the indicated dosing protocol, occur at some point during the course of treatment.

The duration of time can be various time periods. For example, Compound A² and Compound B² can both be administered within a specified period for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 consecutive days during the course of treatment, in which case the duration of time will be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, respectively. When, during the course of treatment, both compounds are administered within a specified period for over 30 consecutive days, the treatment is considered chronic treatment and will continue until an altering event, such as a reassessment in breast cancer status or a change in the condition of the patient, warrants a modification to the protocol.

Various treatment protocols are contemplated in embodiments of the present invention. For example, Compound A² and B² can be co-administered within a specified period for at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days, followed by the administration of Compound A² alone for at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days, in which case the duration of time will be at least the number of consecutive days that Compound A² and Compound B² are both administered plus the number of consecutive days of administration of Compound A² alone (e.g., if Compound A² and Compound B² are both administered for 6 consecutive days followed by administration of Compound A² alone for 8 consecutive days, the duration of time will be at least 14 consecutive days).

In other embodiments, Compound A² and Compound B² are both administered within a specified period for a number of consecutive days during a certain time period, and compound A² is administered during the other days of the certain time period. In some embodiments, the certain time period is n =2, 3, 4, 5, 6, 7, 8, 9, 10, 11, 12, 13, 14, 15, 16, 17, 18, 19, 20, 21, 22, 23, 24, 25, 26, 27, 28, 29 or 30 days, the days of consecutive administration of Compound A² and Compound B² within a specified time period is m =1, 2, 3, 4, 5, 6, 7, 8, 9, 10, 11, 12, 13, 14, 15, 16, 17, 18, 19, 20, 21, 22, 23, 24, 25, 26, 27, 28 or 29, and the days of administration of Compound A² is n-m, where n-m is at least 1. For example, Compound A² and Compound B² can be administered within a specified time period for 1, 2, 3, 4, 5, 6, 7, 8, 9, 10, 11, 12 or 13 consecutive days over a certain time period of 14 days, during which Compound A² is administered for the other 13, 12, 11, 10, 9, 8, 7, 6, 5, 4, 3, 2 or 1 days, respectively. In this example, n=14, m=1, 2, 3, 4, 5, 6, 7, 8, 9, 10, 11, 12 or 13, and n-m=13, 12, 11, 10, 9, 8, 7, 6, 5, 4, 3, 2 or 1, respectively. The consecutive days during which Compound A² and Compound B² are both administered within a specified time period can occur any time during the certain time period. Accordingly, in the foregoing example, Compound A² could be administered alone for 4 consecutive days follow by administration of both Compound A² and Compound B² for 5 consecutive days, followed by administering Compound A² alone for 5 consecutive days to complete the 14 day certain time period.

While treatment protocols have been described with respect to administration of both Compound A² and Compound B² within a specified period in conjunction with administration of Compound A² alone, embodiments of the present invention also include similar treatment protocols in which Compound A² and Compound B² are both administered within a specified period in conjunction with administration of Compound B² alone.

Other embodiments of the present invention include administration of both Compound A² and Compound B² within a specified period in conjunction with administration of Compound A² alone and administration of Compound B² alone. For example, in some embodiments Compound A² and Compound B² are both administered within a specified period for a number of consecutive days during a certain time period, Compound A² is administered alone during a number of days during the certain time period, and Compound B² is administered alone during the other days during the certain time period. In some embodiments, the certain time period is n=3, 4, 5, 6, 7, 8, 9, 10, 11, 12, 13, 14, 15, 16, 17, 18, 19, 20, 21, 22, 23, 24, 25, 26, 27, 28, 29 or 30 days, the days of consecutive administration of Compound A² and Compound B² within a specified time period is m=1, 2, 3, 4, 5, 6, 7, 8, 9, 10, 11, 12, 13, 14, 15, 16, 17, 18, 19, 20, 21, 22, 23, 24, 25, 26, 27 or 28, the days of administration of Compound A² during the certain time period is p=1, 2, 3, 4, 5, 6, 7, 8, 9, 10, 11, 12, 13, 14, 15, 16, 17, 18, 19, 20, 21, 22, 23, 24, 25, 26, 27 or 28, and the days of administration of Compound B² is n-m-p, where n-m-p is at least 1. For example, Compound A² and Compound B² can both be administered within a specified time period for 1, 2, 3, 4, 5, 6, 7, 8, 9, 10, 11 or 12 consecutive days over a certain time period of 14 days, during which Compound A² is administered for 1, 2, 3, 4, 5, 6, 7, 8, 9, 10, 11 or 12 days, and Compound B² is administered for 1, 2, 3, 4, 5, 6, 7, 8, 9, 10, 11 or 12 days. In this example, n=14, m=1, 2, 3, 4, 5, 6, 7, 8, 9, 10, 11 or 12, p=1, 2, 3, 4, 5, 6, 7, 8, 9, 10, 11 or 12, and n-m-p=1, 2, 3, 4, 5, 6, 7, 8, 9, 10, 11 or 12. The consecutive days during which Compound A² and Compound B² are both administered within a specified time period can occur any time during the certain time period. Accordingly, in the foregoing example, Compound A² could be administered alone for 4 consecutive days follow by administration of both Compound A² and Compound B² for 5 consecutive days, followed by administering Compound B² alone for 5 consecutive days to complete the 14 day certain time period. Administration of Compound A² alone and administration of Compound B² alone do not have to occur on consecutive days. Accordingly, in the foregoing example, Compound A² could be administered for 2 consecutive days, followed by administration of Compound B² for 1 day followed by administration of both Compound A² and Compound B² for 5 consecutive days, followed by administration of Compound A² for 1 day, followed by administration of Compound B² for 5 consecutive days.

If the compounds are not administered during a “specified period”, they are administered sequentially. By the term “sequential administration”, and derivatives thereof, as used herein is meant that one of Compound A² and Compound B² is administered for one or more consecutive days and the other of Compound A² and Compound B² is subsequently administered for one or more consecutive days. Also, contemplated herein is a drug holiday utilized between the sequential administration of one of Compound A² and Compound B² and the other of Compound A² and Compound B². As used herein, a drug holiday is a period of one or more days after the administration of one of Compound A² and Compound B² and before the sequential administration of the other of Compound A² and Compound B² where neither Compound A² nor Compound B² is administered. The drug holiday can be a various number of days. In some embodiments, the drug holiday is 1, 2, 3, 4, 5, 6, 7, 8, 9, 10, 11, 12, 13 or 14 days.

In some embodiments, one of Compound A² and Compound B² is administered for 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 consecutive days, followed by an optional drug holiday of 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days, followed by administration of the other of Compound A² and Compound B² for 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 consecutive days.

It is understood that a “specified period” administration and a “sequential” administration can be followed by repeat dosing or can be followed by an alternate dosing protocol, and a drug holiday may precede the repeat dosing or alternate dosing protocol.

It is to be understood that the treatment protocols and regimens described herein can comprise the entire treatment protocol for a given patient or, alternatively, can comprise only a portion of the entire treatment protocol for the patient.

Suitably, the amount of Compound A² administered as part of the combination according to the present invention will be an amount selected from a lower limit of about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295 or 300 mg to an upper limit of about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, or 800 mg. It is to be understood that embodiments of the present invention include any number in the ranges listed above. In some embodiments, the selected amount of Compound A² is administered from 1, 2, 3, 4, 5, or 6 times a day. Suitably, the amount of Compound B² administered as part of the combination according to the present invention will be an amount selected from a lower limit of about 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95 or 2.0 mg to an upper limit of about 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, 2.0, 2.05, 2.10, 2.15, 2.20, 2.25, 2.30, 2.35, 2.40, 2.45, 2.50, 2.55, 2.60, 2.65, 2.70, 2.75, 2.80, 2.85, 2.90, 2.95, 3.0, 3.05, 3.10, 3.15, 3.20, 3.25, 3.30, 3.35, 3.40, 3.45, 3.50, 3.55, 3.60, 3.65, 3.70, 3.75, 3.80, 3.85, 3.90, 3.95 or 4.0 mg. In some embodiments, the selected amount of Compound B² is administered 1, 2, 3, 4, 5 or 6 times a day.

As used herein, all amounts specified for Compound A² and Compound B² are indicated as the administered amount of free or unsalted compound per dose.

The method of the present invention may also be employed with other therapeutic methods of breast cancer treatment.

While it is possible that, for use in therapy, therapeutically effective amounts of the combinations of the present invention may be administered as the raw chemical, it is preferable to present the combinations as a pharmaceutical composition or compositions. Accordingly, the invention further provides pharmaceutical compositions, which include Compound A² and/or Compound B², and one or more pharmaceutically acceptable carriers for the treatment of breast cancer. The combinations of the present invention are as described above. The carrier(s) must be acceptable in the sense of being compatible with the other ingredients of the formulation, capable of pharmaceutical formulation, and not deleterious to the recipient thereof. In accordance with another aspect of the invention there is also provided a process for the preparation of a pharmaceutical formulation for the treatment of breast cancer including admixing Compound A² and/or Compound B² with one or more pharmaceutically acceptable carriers. As indicated above, such elements of the pharmaceutical combination utilized may be presented in separate pharmaceutical compositions or formulated together in one pharmaceutical formulation.

Pharmaceutical formulations may be presented in unit dose forms containing a predetermined amount of active ingredient per unit dose. As is known to those skilled in the art, the amount of active ingredient per dose will depend on the condition being treated, the route of administration and the age, weight and condition of the patient. Preferred unit dosage formulations are those containing a daily dose or sub-dose, or an appropriate fraction thereof, of an active ingredient. Furthermore, such pharmaceutical formulations may be prepared by any of the methods well known in the pharmacy art.

Compound A² and Compound B² may be administered by any appropriate route. Suitable routes include oral, rectal, nasal, topical (including buccal and sublingual), vaginal, and parenteral (including subcutaneous, intramuscular, intravenous, intradermal, intrathecal, and epidural). It will be appreciated that the preferred route may vary with, for example, the condition of the recipient of the combination and the precise nature of the breast cancer to be treated. It will also be appreciated that each of the agents administered may be administered by the same or different routes and that Compound A² and Compound B² may be compounded together in a pharmaceutical composition/formulation. In some embodiments, Compound A² and Compound B² are administered in separate pharmaceutical compositions. In other embodiments, Compound A² and Compound B² are administered in fixed-dose pharmaceutical compositions that include both Compound A² and Compound B².

The compounds or combinations of the current invention are incorporated into convenient dosage forms such as capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid. Liquid carriers include syrup, peanut oil, olive oil, saline, and water. Similarly, the carrier may include a prolonged release material, such as glyceryl monostearate or glyceryl distearate, alone or with a wax. The amount of solid carrier varies widely but, suitably, may be from about 0.05 mg to about 1 g per dosage unit. When a liquid carrier is used, the preparation will suitably be in the form of a syrup, elixir, emulsion, soft gelatin capsule, sterile injectable liquid such as an ampoule, or an aqueous or nonaqueous liquid suspension.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

It should be understood that in addition to the ingredients mentioned above, the formulations may include other agents conventional in the art having regard to the type of formulation in question, for example those suitable for oral administration may include flavoring agents.

As indicated, therapeutically effective amounts of the combinations of the invention (Compound A² in combination with Compound B²) are administered to a human. In some embodiments, the human is a female. Typically, the therapeutically effective amount of the administered agents of the present invention will depend upon a number of factors including, for example, the age and weight of the subject, the precise condition requiring treatment, the severity of the condition, the nature of the formulation, and the route of administration. Ultimately, the therapeutically effective amount will be at the discretion of the attending physician.

This invention also provides for a combination comprising 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof and (S)-10-[(dimethylamino)methyl]-4-ethyl-4,9-dihydroxy-1H-pyrano[3′,4′:6,7]indolizino[1,2-b]quinoline-3,14-(4H,12H)-dione, or a pharmaceutically acceptable salt, suitably the hydrochloride salt, thereof, for use in the treatment of breast cancer.

This invention also provides for the use of a combination comprising 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof and (S)-10-[(dimethylamino)methyl]-4-ethyl-4,9-dihydroxy-1H-pyrano[3′,4′:6,7]indolizino[1,2-b]quinoline-3,14-(4H,12H)-dione, or a pharmaceutically acceptable salt, suitably the hydrochloride salt, thereof, in the manufacture of a medicament for the treatment of breast cancer.

This invention also provides a method of treating breast cancer which comprises administering a combination of 5-[[4-(2,3-dimethyl-2H-indazol-6-yl)methylamino]-2-pyrimidinyl]amino]-2-methylbenzenesulfonamide, or a pharmaceutically acceptable salt, suitably the monohydrochloride salt, thereof and (S)-10-[(dimethylamino)methyl]-4-ethyl-4,9-dihydroxy-1H-pyrano[3′,4′:6,7]indolizino[1,2-b]quinoline-3,14-(4H,12H)-dione, or a pharmaceutically acceptable salt, suitably the hydrochloride salt, thereof, to a subject in need thereof.

The following examples are intended for illustration only and are not intended to limit the scope of the invention in any way.

### Experimental Details

### Materials and Methods:

### Drugs and Reagents:

Topotecan hydrochloride, (S)-10-[(dimethylamino)methyl]-4-ethyl-4,9-dihydroxy-1H-yrano[3′,4′:6,7]indolizino[1,2-b]quinoline-3,14(4H,12H)-dione monohydrochloride and pazopanib monohydrochloride, (5-[[4-[(2,3-Dimethyl-2H-indazol-6-yl)methylamino]-2-pyrimidinyl]amino]-2-methylbenzolsulfonamide are available from GlaxoSmithkline. Topotecan and pazopanib were obtained from GlaxoSmithKline. Topotecan used for MTD schedule was manufactured by Sandoz Canada Inc, QC, Canada, and purchased from the institutional pharmacy. All drugs were prepared according to manufacturers' instructions.

### Cell Lines:

The original cell line, MDA-MB 231, was a gift from Dr. Jeffrey F. Lemontt in September, 1989, together with other variants of this cell line. Cell line 231/LM2-4 (human breast cancer cells) used in the study was derived by one of the inventors as described in Munoz R, Man S, Shaked Y, et al. Highly efficacious non-toxic treatment for advanced metastatic breast cancer using combination UFT-cyclophosphamide metronomic chemotherapy, Cancer Res 2006; 66:3386-91.

### Metastatic Model:

Two million 231/LM2-4 human breast cancer cells were implanted into the mammary fat pad of 6 -8 week old female CB-17 SCID mice purchased from Charles River Canada. Twenty days later when tumor volumes were approximately 400 mm³, the primary tumors were surgically resected. Therapy was initiated 19 days after resection when visceral metastases were established in sites such as lung and liver. Mice were randomized into groups of 4 and treated as follows: 1) Control−vehicle; 2) Low dose metronomic oral topotecan; 3) pazopanib; 4) Low dose metronomic oral topotecan+pazopanib; 5) Maximum tolerated dose topotecan; and 6) maximum tolerated dose topotecan+pazopanib. The schedule and doses of drugs used were metronomic oral topotecan 1 mg/kg/d by gavage, maximum tolerated dose topotecan 1.5 mg/kg by gavage for 5 consecutive days followed by 16 days break ip, pazopanib 150 mg/kg/d by gavage, and control mice were given the topotecan vehicle and/or pazopanib vehicle. The oral topotecan and pazopanib preparations were mixed just prior to administration to avoid possible drug interaction. Institutional guidelines for survival endpoint were followed.

### Results:

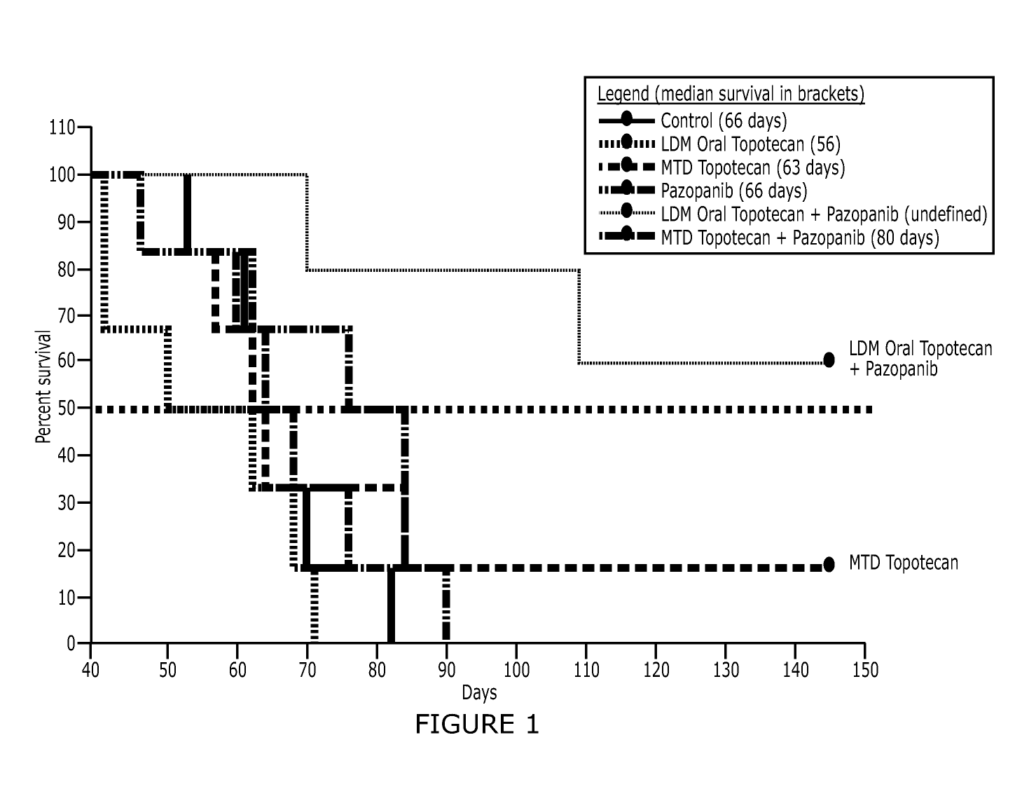

As shown in FIG. 1, neither low dose metronomic (LDM) oral topotecan alone nor pazopanib alone had any survival benefit when compared to the control untreated mice. One mouse in the LDM oral topotecan+pazopanib treated group was euthanized early in the experiment due to bowel obstruction and this was taken out of the results. Median survival for control was 66 days, for LDM oral topotecan alone was 56 days, for pazopanib alone was 66 days, for maximum tolerated dose (MTD) topotecan was 63 days and for MTD topotecan+pazopanib was 80 days, whereas more than 50% of mice treated with LDM oral topotecan in combination with pazopanib were still alive at 150 days.

While the preferred embodiments of the invention are illustrated by the above, it is to be understood that the invention is not limited to the precise instructions herein disclosed and that the right to all modifications coming within the scope of the following claims is reserved.

What is claimed is:

1. A combination comprising:
(i) a compound of Structure (I): 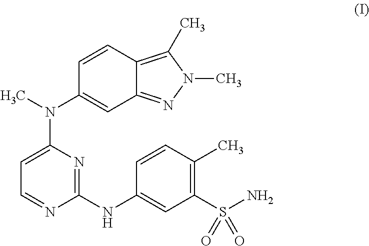 [SMILES: Cc1ccc(Nc2nccc(N(C)c3ccc4c(C)n(C)nc4c3)n2)cc1S(N)(=O)=O]
or a pharmaceutically acceptable salt thereof; and
(ii) a compound of Structure (II): 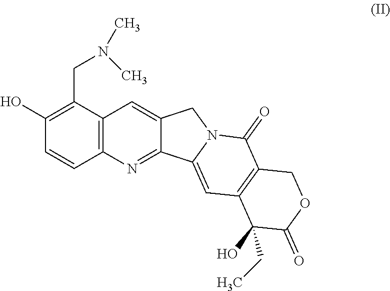 [SMILES: CC[C@@]1(O)C(=O)OCc2c1cc1n(c2=O)Cc2cc3c(CN(C)C)c(O)ccc3nc2-1]
or a pharmaceutically acceptable salt thereof.

2. A combination according to claim 1 where the compounds of Structure (I) and Structure (II) are each in the form of a monohydrochloride salt.

3. A combination according to claim 1 where the amount of the compound of Structure (I) is an amount from 5 mg to 800 mg, and that amount is administered once per day in one or more tablets, and the amount of the compound of Structure (II) is an amount from 0.05 mg to 1 mg, and that amount is administered once per day.

4. (canceled)

5. A method of treating breast cancer in a human in need thereof, comprising the in vivo administration of a therapeutically effective amount of a combination of 5-[[4-[(2,3-dimethyl-2H-indazol-6-yl)methylamino]-2-pyrimidinyl]amino]-2-methylbenzenesulfonamide, or a pharmaceutically acceptable salt thereof, and (S)-10-[(dimethylamino)methyl]-4-ethyl-4,9-dihydroxy-1H-pyrano[3′,4′:6,7]indolizino[1,2-b]quinoline-3,14-(4H,12H)-dione, or a pharmaceutically acceptable salt thereof, to such human, wherein the combination is administered within a specified period, and wherein the combination is administered for a duration of time.

6. A method according to claim 5 wherein the amount of 5-[[4-[(2,3-dimethyl-2H-indazol-6-yl)methylamino]-2-pyrimidinyl]amino]-2-methylbenzenesulfonamide, or a pharmaceutically acceptable salt thereof, is from about 5 mg to about 800 mg, and that amount is administered once per day.

7. A method according to claim 5, wherein the amount of (S)-10-[(dimethylamino)methyl]-4-ethyl-4,9-dihydroxy-1H-pyrano[3′,4′:6 ,7]indolizino[1,2-b]quinoline-3,14-(4H,12H)-dione, or a pharmaceutically acceptable salt thereof, is from about 0.05 mg to about 1 mg, and that amount is administered once per day.

8. A method according to claim 5 wherein the specified period is within about 1 to about 12 hours.

9. A method according to claim 5 wherein the duration of time is for from 1 to 30 consecutive days.

10. A method according to claim 5 wherein the human is female.

11. A method according to any claim 5 wherein 5-[[4-[(2,3-dimethyl-2H-indazol-6-yl)methylamino]-2-pyrimidinyl]amino]-2-methylbenzenesulfonamide and (S)-10-[(dimethylamino)methyl]-4-ethyl-4,9-dihydroxy-1H-pyrano[3′,4′:6,7]indolizino[1,2-b]quinoline-3,14-(4H,12H)-dione are each in the form of a monohydrochloride salt.

12. A method treating breast cancer in a human in need thereof, comprising the in vivo administration of a therapeutically effective amount of a combination of 5-[[4-[(2,3-dimethyl-2H-indazol-6-yl)methylamino]-2-pyrimidinyl]amino]-2-methylbenzenesulfonamide, or a pharmaceutically acceptable salt thereof, and (S)-10-[(dimethylamino)methyl]-4-ethyl-4,9-dihydroxy-1H-pyrano[3′,4′:6,7]indolizino[1,2-b]quinoline-3,14-(4H,12H)-dione, or a pharmaceutically acceptable salt thereof, to such human, wherein the compounds of the combination are administered sequentially.

13. A method according to claim 12, wherein the amount of 5-[[4-[(2,3-dimethyl-2H-indazol-6-yl)methylamino]-2-pyrimidinyl]amino]-2-methylbenzenesulfonamide, or a pharmaceutically acceptable salt thereof, is from about 5 mg to about 800 mg, and that amount is administered once per day.

14. A method according to claim 12 or 13, wherein the amount of (S)-10-[(dimethylamino)methyl]-4-ethyl-4,9-dihydroxy-1 H-pyrano[3′,4′:6 ,7]indolizino[1,2-b]quinoline-3,14-(4H,12H)-dione, or a pharmaceutically acceptable salt thereof, is from about 0.05 mg to about 1 mg, and that amount is administered once per day.

15. A method according to claim 12 wherein 5-[[4-[(2,3-dimethyl-2H-indazol-6-yl)methylamino]-2-pyrimidinyl]amino]-2-methylbenzenesulfonamide, or a pharmaceutically acceptable salt thereof, is administered for from 1 to 30 consecutive days, followed by an optional drug holiday of from 1 to 14 days, followed by administration of (S)-10-[(dimethylamino)methyl]-4-ethyl-4,9-dihydroxy-1H-pyrano[3′,4′:6,7]indolizino[1,2-b]quinoline-3,14-(4H,12H)-dione, or a pharmaceutically acceptable salt thereof for from 1 to 30 days.

16. A method according to claim 12 wherein the human is female.

17. A method according to claim 12 wherein 5-[[4-[(2,3-dimethyl-2H-indazol-6-yl)methylamino]-2-pyrimidinyl]amino]-2-methylbenzenesulfonamide and (S)-10-[(dimethylamino)methyl]-4-ethyl-4,9-dihydroxy-1H-pyrano[3′,4′:6,7]indolizino[1,2-b]quinoline-3,14-(4H,12H)-dione are each in the form of a monohydrochloride salt.

18. A combination comprising 5-[[4-[(2,3-dimethyl-2H-indazol-6-yl)methylamino]-2-pyrimidinyl]amino]-2-methylbenzenesulfonamide, or a pharmaceutically acceptable salt thereof, and (S)-10-[(dimethylamino)methyl]-4-ethyl-4,9-dihydroxy-1H-pyrano[3′,4′:6,7]indolizino[1,2-b]quinoline-3,14-(4H,12H)-dione, or a pharmaceutically acceptable salt thereof for use in the treatment of breast cancer.

19. A combination according to claim 18, wherein the amount of 5-[[4-[(2,3-dimethyl-2H-indazol-6-yl)methylamino]-2-pyrimidinyl]amino]-2-methylbenzenesulfonamide, or a pharmaceutically acceptable salt thereof, is from about 5 mg to about 800 mg.

20. A combination according to claim 18 or 19, wherein the amount of (S)-10-[(dimethylamino)methyl]-4-ethyl-4,9-dihydroxy-1H-pyrano[3′,4′:6,7]indolizino[1,2-b]quinoline-3,14-(4H,12H)-dione, or a pharmaceutically acceptable salt thereof, is from about 0.05 mg to about 1 mg.

21. A combination according to claim 18 wherein 5-[[4-[(2,3-dimethyl-2H-indazol-6-yl)methylamino]-2-pyrimidinyl]amino]-2-methylbenzenesulfonamide and (S)-10-[(dimethylamino)methyl]-4-ethyl-4,9-dihydroxy-1H-pyrano[3′,4′:6,7]indolizino[1,2-b]quinoline-3,14-(4H,12H)-dione are each in the form of a monohydrochloride salt.

22. (canceled)
